(19) United States
(12) Patent Application Publication
(10) Pub. No.: US 2014/0177263 A1
(43) Pub. Date: Jun. 26, 2014

(54) ELECTRONIC INCENSE AND ELECTRONIC CENSER WITH THE SAME

(71) Applicant: HON HAI PRECISION INDUSTRY CO., LTD., New Taipei (TW)

(72) Inventor: CHIH-CHEN LAI, New Taipei (TW)

(73) Assignee: HON HAI PRECISION INDUSTRY CO., LTD., New Taipei (TW)

(21) Appl. No.: 13/949,248

(22) Filed: Jul. 24, 2013

(30) Foreign Application Priority Data
Dec. 24, 2012 (TW) 101149445

(51) Int. Cl. A61L 9/03; F21V 33/00

(52) U.S. Cl. CPC A61L 9/03; F21V 33/0028
USPC 362/555

(57) ABSTRACT

An electronic censer includes a seat and multiple real incenses and an electronic incense inserted to the seat. The electronic incense includes a light guiding rod, a metal shell enclosing the rod, an LED contacting the rod and a circuit board connected to the LED. Light emitted from the LED transmits through the rod to an outside environment.

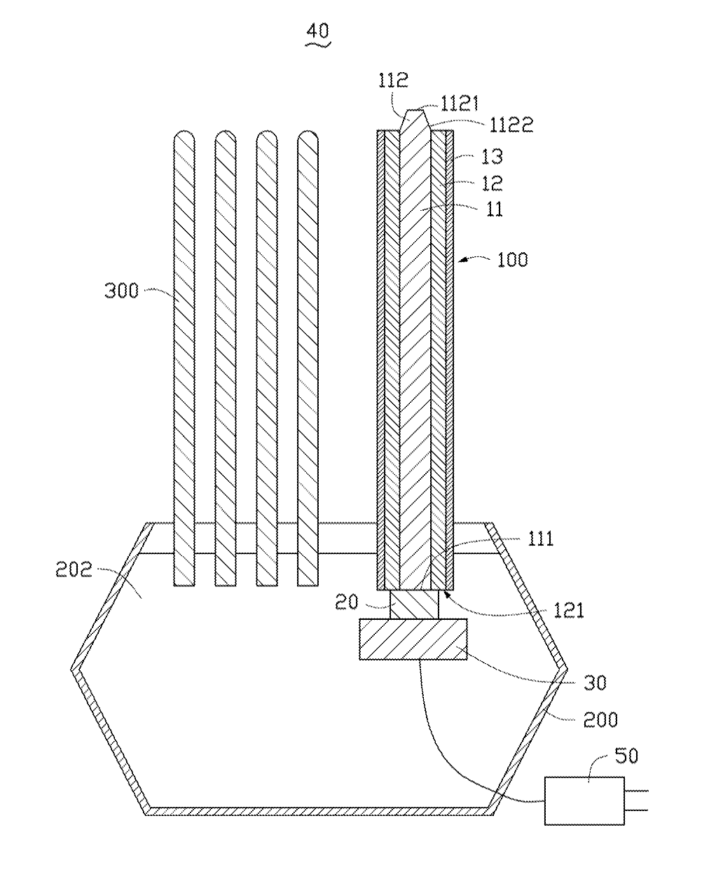

### BACKGROUND

1. Technical Field

The disclosure generally relates to an electronic device, and particularly to an electronic incense and an electronic censer with the heat resistance incense.

2. Description of Related Art

Traditional censers includes seats and real incenses inserted to the seats. A real incense includes a bamboo core and aromatic biotic materials coated on the core. When burned, the aromatic biotic materials release large amounts of smoke. The burning incenses may ignite surrounding articles to fire; furthermore, the released smoke not only pollutes the environment, but also is harmful to health.

Generally, electronic censers are used to replace the traditional censers. An typical electronic censer includes a seat and an electronic incense inserted to the seat. The electronic incense includes a rod, an LED and a protective layer enclosing the rod. Light emitted from the LED is guided by the rod to emit to the outside environment. The protective layer is usually made of plastic material which has a low melting point. Thus, when the electronic incense is used with the real incense in a same seat, the high temperature due to burning of the real incense may damage the protective layer of the electronic incense. Even worse, if the real incense is inclined to contact the electronic incense, the burning tip of the real incense may cause the protective layer of the electronic incense to be burned.

What is needed, therefore, is an electronic incense and an electronic censer with the heat resistance incense which can overcome the described limitations.

### BRIEF DESCRIPTION OF THE DRAWINGS

Many aspects of the present embodiments can be better understood with reference to the following drawing. The components in the drawing are not necessarily drawn to scale, the emphasis instead being placed upon clearly illustrating the principles of the present embodiments. Moreover, in the drawing, like reference numerals designate corresponding parts throughout the several views.

The only drawing shows an electronic censer in accordance with an embodiment of the present disclosure.

### DETAILED DESCRIPTION

Referring to the figure, an electronic censer 40 in accordance with an embodiment of the disclosure is shown. The electronic censer 40 includes a seat 200, an electronic incense 100 and a plurality of real incenses 300 inserted into the seat 200.

The seat 200 has a bowl shape with an opening defined at the top thereof. The seat 200 may be filled with ash 202 to imitate a seat of a traditional censer.

The electronic incense 100 includes a light guiding rod 11, a shell 12 enclosing the rod 11, an LED (light emitting diode) 20 attached on the rod 11 and the shell 12, and a circuit board 30 connecting to the LED 20. The rod 11 may be made of transparent material such as epoxy, glass or the like. In this embodiment, the rod 11 is an optical fiber. The rod 11 is elongated and straight. A bottom face 111 of the rod 11 contacts a top face of the LED 20 for introducing light emitted from the LED 20 to an interior of the rod 11. The rod 11 is mostly received in the shell 12 except a top end thereof. The top end of the rod 11 forms a light emission tip 112 protruding out of the shell 12. The light emission tip 112 has a flat top face 1121 and a conical circumferential face 1122 connecting the top face. The circumferential face 1122 of the light emission tip 112 has a diameter gradually increasing from the top face 1121 of the light emission tip 112 towards the bottom face 111 of the rod 22. The top face 1121 and the circumferential face 1122 of the light emission tip 112 form a large amount of micro grooves or protrusions thereon. Thus, the top face 1121 and the circumferential face 1122 of the light emission tip 112 are rough for uniformly diffusing the light to an outside environment.

The shell 12 encases the rod 11 therein. The shell 12 may be made of heat resistance and rigid material such as metal. Thus, the rod 11 is protected and supported by the shell 12. Even the rod 11 is very thin, the rigid shell 12 can still support the rod 11 to prevent undesired bending of the rod 11. The shell 12 may form a light reflective film (not visible) on an inner circumferential face thereof. Thus, the light can be confined by the light reflective film to transmit within the rod 11. The shell 12 has a bottom face 121 flush with the bottom face 111 of the rod 11, and a top face lower than the top face of the light emission tip 112. The bottom face 121 of the shell 12 directly contacts the top face of the LED 20.

The LED 20 has a light emerging face formed on the top face thereof. The LED 20 may be a red LED 20 which emits red light when activated, thereby imitating burning of the traditional incense.

The circuit board 30 is fixed to and electrically connected to the LED 20. The circuit board 30 is also electrically connected to a plug 50. The plug 50 can be inserted to a general power socket to introduce power to the circuit board 30. The circuit board 30 then drives the LED 20 to lighten after receiving the power from the plug 50.

A coating layer 13 is further formed on an outer circumferential face of the shell 12. The coating layer 13 may be made of heat resistance paint. The coating layer 13 may have a color similar to the traditional incense in order to imitate the appearance of the traditional incense. The coating layer 13 has a thickness less than that of the shell 12.

In use, the electronic incense 100 and the real incenses 300 are all inserted to the ash 202 of the seat 200 in a manner than the real incenses 300 are located adjacent to the electronic incense 100. The real incenses 300 are ignited to burn. The LED 20 is activated by the circuit board 30 to emit the red light. Thus, the electronic incense 100 imitates burning of the real incense 300. Since the shell 12 of the electronic incense 100 is heat resisting, the burning of the real incenses 300 will not damage the electronic incense 100 even the burning tips of the real incenses 300 directly contacting the electronic incense 100.

It is to be understood, however, that even though numerous characteristics and advantages of the present embodiments have been set forth in the foregoing description, together with details of the structures and functions of the embodiments, the disclosure is illustrative only, and changes may be made in detail, especially in matters of shape, size, and arrangement of parts within the principles of the disclosure to the full extent indicated by the broad general meaning of the terms in which the appended claims are expressed.

What is claimed is:

1. An electronic censer comprising:
a seat; and
an electronic incense inserted into the seat, the electronic incense comprising:
a light guiding rod;
a heat resistance shell enclosing the light guiding rod; and
a light emitting diode facing the light guiding rod, light emitted from the light emitting diode exiting out of a top of the light guiding rod after passing through the light guiding rod.

2. The electronic censer of claim 1, wherein the seat is containing ash, and the electronic incense is inserted into the ash.

3. The electronic censer of claim 1, wherein the light guiding rod comprises a light emission tip protruding beyond a top of the shell, the light emission tip diffusing the light to the outside environment of the electronic censer.

4. The electronic censer of claim 3, wherein the light emission tip comprises a flat top face and a conical circumferential face connecting the flat top face, the flat top face and the conical circumferential face being rough.

5. The electronic censer of claim 1, wherein the light guiding rod comprises a bottom face directly contacting a top face of the light emitting diode.

6. The electronic censer of claim 5, wherein the shell comprises a bottom face directly contacting the top face of the light emitting diode.

7. The electronic censer of claim 6, wherein the bottom face of the shell is flush with that of the light guiding rod.

8. The electronic censer of claim 1, wherein the electronic incense further comprises a coating layer formed on an outer circumferential face of the shell.

9. The electronic censer of claim 8, wherein the coating layer is made of heat resistance paint.

10. The electronic censer of claim 8, wherein the coating layer has a thickness less than that of the shell.

11. The electronic censer of claim 8 further comprising a real incense inserted into the seat, wherein the coating layer of the electronic incense has a color similar to that of the real incense.

12. The electronic censer of claim 11, wherein the real incense is located adjacent to the electronic incense.

13. The electronic censer of claim 1, wherein the electronic incense further comprises a circuit board fixed to the light emitting diode.

14. The electronic censer of claim 13, wherein the circuit board electrically connects the light emitting diode with a plug.

15. The electronic censer of claim 1, wherein the shell is made of rigid material.

16. The electronic censer of claim 1, wherein the shell is made of metal.

17. The electronic censer of claim 1, wherein the light guiding rod is made of transparent material.

18. The electronic censer of claim 1, wherein the light guiding rod is an optical fiber.

19. An electronic incense comprising:
a light guiding rod;
a heat resistance shell enclosing the light guiding rod; and
a light emitting diode facing the light guiding rod, light emitted from the light emitting diode being guided by the light guiding rod to exit out of the electronic incense.

20. The electronic incense of claim 20, wherein the shell is made of metal.
